(19) United States
(12) Patent Application Publication
(10) Pub. No.: US 2023/0395252 A1
(43) Pub. Date: Dec. 7, 2023

(54) Patient Monitor System for Collecting Data of a Patient, Display Device, Medical Treatment Apparatus and Method

(71) Applicant: Fresenius Medical Care Deutschland GmbH, Bad Homburg (DE)

(72) Inventors: David Hannes, Frankfurt/Main (DE); Martin Urban, Güntersleben (DE); Christoph Wiktor, Gelnhausen (DE)

(21) Appl. No.: 18/033,883

(22) PCT Filed: Oct. 27, 2021
(86) PCT No.: PCT/EP2021/079830

§ 371 Date: Apr. 26, 2023

(30) Foreign Application Priority Data
Oct. 28, 2020 (DE) 10 2020 128 298.2

(51) Int. Cl. G16H 40/67; G16H 10/60; G16H 50/70; G16H 50/30; G16H 20/40; A61M 1/34; A61M 1/28; A61M 1/16

(52) U.S. Cl. CPC G16H 40/67; G16H 10/60; G16H 50/70; G16H 50/30; G16H 20/40; A61M 1/3496; A61M 1/28; A61M 1/1601

(57) ABSTRACT

The present disclosure relates to a patient monitor system with at least one collecting device which is programmed to collect behavior-related data and/or vital parameters of a patient. The patient monitor system further includes an output device and a calculation unit. The calculation unit is programmed to prompt or trigger the collecting device in order to collect the data and vital parameters. It is further programmed to generate instructions for controlling or regulating a medical treatment apparatus, and for outputting the instructions by the output device. The calculation unit is hereby further programmed to generate instructions and/or to have them output by the output device when the behavior-related data of the patient, or part of this data, and/or the vital parameters of the patient, or part these vital parameters do not lie in a value range respectively predetermined for them and/or lie beyond a limit value respectively predetermined for them.

### CROSS-REFERENCE TO RELATED APPLICATIONS

The present application is the national stage entry of International Application No. PCT/EP2021/079830, filed on Oct. 27, 2021, and claims priority to Application No. DE 102020128298.2, filed in the Federal Republic of Germany on Oct. 28, 2020, the disclosures of which are expressly incorporated herein in their entirety by reference thereto.

### TECHNICAL FIELD

The present disclosure relates to a patient monitor system as described herein, a display device as described herein, and a medical treatment apparatus as described herein. The disclosure also relates to a method for preparing a treatment as described herein.

### BACKGROUND

Various types of medical treatment apparatuses are known. They include blood treatment apparatuses for extracorporeal blood treatment, for example the apparatuses for hemodialysis, hemofiltration and hemodiafiltration.

During the extracorporeal blood treatment, the blood flows in an extracorporeal blood circuit through a blood treatment unit. In the apparatuses for hemodialysis, hemofiltration and hemodiafiltration, the blood treatment unit is a dialyzer or filter which, in simple terms, is separated into a blood chamber and a dialysis liquid chamber by a semipermeable membrane. During the blood treatment by hemodialysis or hemodiafiltration, the blood flows through the blood chamber, while a dialysis liquid flows through the dialysis liquid chamber.

Since extracorporeal blood treatment may be stressful for the patient, blood treatment parameters are individually adapted at the blood treatment apparatus to the patient and their needs, but also to their daily form, which may be checked by measuring parameters or data of the patient.

The present disclosure proposes a patient monitor system for detecting data or parameters of the patient. Furthermore, a display device and a medical treatment apparatus are to be proposed. In addition, a method for preparing a treatment is to be specified.

### SUMMARY

According to the present disclosure, a patient monitor system is proposed which includes at least one collecting device or detecting device. It is configured and/or programmed to collect or measure behavior-related data of a patient on the one hand and/or vital parameters of the patient on the other.

Furthermore, the patient monitor system includes a calculation unit which is programmed to receive behavior-related data and/or vital parameters of the patient, collected via the collecting device, automatically or upon request, e.g., by the patient.

The calculation unit is further programmed to generate instructions which can be used for direct or indirect control or regulation of a medical treatment apparatus or which are instructions for carrying out, adjusting, adapting, etc., the control or regulation of the medical treatment apparatus.

The instructions can be control signals or commands in machine language for the medical treatment apparatus to control or regulate it during an ongoing and/or at least during an upcoming treatment session by the medical treatment apparatus.

Likewise, instructions or directions for the attending physician to adjust treatment parameters on the medical treatment apparatus, or with effect for the same, are encompassed as instructions by the present disclosure.

Instructions may be issued with the aim of adapting the parameters of a treatment or treatment session and/or changing treatment parameters already stored or collected and/or adapted in previous treatment sessions.

The calculation unit is further programmed to output the instructions by an output device, which is also encompassed by the patient monitor system.

Furthermore, the calculation unit is programmed to generate and/or to enable outputting instructions by the output device then or only then if the collected behavior-related data of the patient, or a part of these data, and the collected vital parameters of the patient, or a part of these vital parameters, do not lie in a value range predetermined for them and/or lie beyond a limit value predetermined for them.

According to the present disclosure, a display device for displaying one or more instructions for controlling or regulating a medical treatment apparatus is further proposed. The display device is in signal communication with the output device of the patient monitor system. It may be part of a patient monitor system described herein or is provided externally thereto, for example in its own housing. The display device may thus be integrated, for example, in the medical treatment apparatus described herein. Alternatively or additionally, the display device may be designed, for example, on a monitoring station for a plurality of treatment apparatuses. Alternatively or additionally, the display device may be a tablet, smartphone, or other screen that displays the generated instructions to the attending physician.

According to the present disclosure, a medical treatment apparatus is further proposed, which includes a patient monitor system as described herein and/or a display device as described herein or is connected thereto, e.g., in a wired, wireless or in any other way, and/or is in signal communication therewith or is prepared for this.

When reference is made herein to a signal communication or communication connection between two components, this may be understood to mean a connection that exists in use. Likewise, it may be understood herein that there is a preparation for such a signal communication (wired, wireless, or otherwise implemented), for example, by a coupling of both components, such as by pairing, etc.

Pairing is to be understood as a process that takes place in connection with computer networks in order to establish an initial link between computer units for the purpose of communication. The best-known example of this is the establishment of a Bluetooth connection, by which various devices (e.g., smartphone, headphones) are connected to one another. Pairing is sometimes referred to as bonding.

Furthermore, according to the present disclosure, a method for preparing a treatment or treatment session of a patient is specified, wherein the treatment or treatment session is to be carried out by using a medical treatment apparatus.

The preparation of a treatment takes place, for example, prior to a treatment or treatment session, i.e., before the patient is connected to the treatment apparatus or to an extracorporeal blood circuit thereof. The method includes:
a. collecting behavior-related data at at least one point in time at which the patient is not being treated by the medical treatment apparatus in a treatment session, or between two successive treatments or treatment sessions which are, or have been, performed by using the medical treatment apparatus. The behavior-related data, as disclosed herein, may include, for example, data on sleep behavior, movement data, in particular the number of steps (possibly based on specific time units), data on regular medication intake and the like. The behavior-related data may be collected automatically by sensors or manually through input by the patient.
b. collecting vital parameters at at least one point in time, in particular the aforementioned period of time, at which the patient is not being treated by the medical treatment apparatus in a treatment session or between two successive treatments or treatment sessions which are, or have been, performed by the medical treatment apparatus. Vital parameters, as disclosed herein, may include, for example, cardiovascular data (HR, HRV, QT wave, etc.), weight, temperature, and the like. The vital parameters may be collected automatically by sensors or manually through input by the patient.
c. Outputting instructions when the behavior-related data of the patient and/or the vital parameters of the patient are not in a value range predetermined for them and/or lie beyond a limit value predetermined for them.

Optionally, the method described above may be carried out in the above-mentioned order, overlapping in time or simultaneously.

In all of the following statements, the use of the expression “may be” or “may have” and so on, is to be understood synonymously with “preferably is” or “preferably has” and so on, respectively, and is intended to illustrate an embodiment according to the present disclosure.

Whenever numerical words are mentioned herein, the person skilled in the art shall recognize or understand them as indications of a numerical lower limit. Unless it leads the person skilled in the art to an evident contradiction, the person skilled in the art shall comprehend the specification of for example “one” as encompassing “at least one”. This understanding is also equally encompassed by the present disclosure as the interpretation that a numerical word, for example, “one” may alternatively mean “exactly one”, wherever this is evidently technically possible for the person skilled in the art. Both understandings are encompassed by the present disclosure and apply to all numerical words used herein.

Whenever “programmed” or “configured” is mentioned herein, it is hence also disclosed that these terms are to be interchangeable.

Whenever an embodiment is mentioned herein, it is then an exemplary embodiment according to the present disclosure.

When it is disclosed herein that the subject-matter according to the present disclosure includes one or several features in a certain embodiment, it is also respectively disclosed herein that the subject-matter according to the present disclosure does, in other embodiments, likewise according to the present disclosure, explicitly not include this or these features, for example, in the sense of a disclaimer. Therefore, for every embodiment mentioned herein it applies that the converse embodiment, e.g., formulated as negation, is also disclosed.

Embodiments according to the present disclosure may include one or more of the features mentioned above and/or below in any technically possible combination.

Whenever a suitability or a method is mentioned herein, the present disclosure also encompasses corresponding programming or configuration of a suitable apparatus or a section thereof.

Instructions may include, for example, a request to adjust the ultrafiltration rate in order to reduce the likelihood of heart failure, high blood pressure or sleep apnea. They may optionally include adjustment of a medication used during blood treatment or dialysis treatment such that the likelihood of hyperkalemia is reduced, for example, by affecting acid-base balance.

In several embodiments of the patient monitor system described herein, the calculation unit is programmed to prompt the behavior-related data and/or vital parameters to be collected by the collecting device. This takes place preferably at least at one point in time and optionally only when the patient is not being treated by the medical treatment apparatus in a treatment session, or optionally only between two successive treatment sessions at which the patient was or would have been treated by the medical treatment apparatus.

In some embodiments, the calculation unit is programmed to collect the vital parameters, or a part thereof, or to evaluate them respectively only in the event that the calculation unit has recognized that the collected behavior-related data, or a part thereof, is not in a value range respectively predetermined for them and/or lie beyond a limit value respectively predetermined for them.

In several embodiments, the collecting device preferably includes at least one sensor or a plurality of sensors, i.e., two or more, by which the collection or measurement of values may be performed automatically.

In some embodiments, the collecting device includes, alternatively or additionally, an input device by which values may be entered manually, for example by the patient.

In other embodiments, the reverse is true. The calculation unit is programmed to collect the behavior-related data, or a part thereof, or to evaluate it respectively only in the event that the calculation unit has recognized that the collected vital parameters, or part of them, are not in a value range respectively predetermined for them and/or lie beyond a limit value respectively predetermined for them. Thus, in the case of poor vital values, the current state of mind, which may lead to e.g., changes in blood pressure or blood rate, can be checked and recognized as a cause that may lead to other adaptations or adjustments of the treatment parameters than vital values that cannot be explained by the patient's current state of mind.

In several embodiments, the collecting device is designed as a portable device, in particular as a wristband, wrist watch, mobile phone (e.g., smartphone, iPhone, etc.), wearable, accessory, as a garment or respectively part thereof. Combinations of the foregoing designs are also encompassed by the present disclosure. For example, parts or components (especially sensors) of the collecting device may be present on, for example, the wristband, while others are provided in, for example, the clothing. Similarly, medication intake and/or activity and/or food intake, blood pressure, etc. may be entered by the patient via a smartphone, while other data, such as pulse, heart rate variability, or body temperature, etc., may be collected via a device worn on the wrist or upper arm.

In certain embodiments it is provided that the patient carries the collecting device with them permanently or for a longer period of time, in particular between two successive treatments, and in particular also in their private environment (at home, at work, during leisure time, etc.).

In some embodiments of the patient monitor system, the behavior-related data are, or include, sleep data, movement data, data on the perception of stress, data on, in particular, regular, medication intake and/or data on the patient's intake of food and/or liquid.

Sleep data may be, or may include, sleep duration, quality of sleep (e.g., whether the patient wakes up frequently), regularity of sleep, and/or analysis of individual sleep phases (falling asleep, light sleep, REM, and deep sleep phases).

Movement data may be, or may include, a number of steps, in particular related to a specific time unit, information from the patient on activities, etc.

Data on the perception of stress may, for example, be assessed individually by the patient and entered on a scale, for example from 1 to 10.

Data on regular medication intake may be, or may include a medication, an active ingredient, a dose and/or a concentration. In certain embodiments, it may be provided to compare the patient's information about the aforementioned with a predetermined and stored therapy plan. Outputs may be provided in case discrepancies or deviations are detected between the collected medication intake and the stored therapy plan. Optionally, the patient monitor system may be programmed to output reminders if, for example, times for taking medications have elapsed without their intake having been confirmed by the patient by a corresponding input via the input device.

Data on food and liquid intake may be or may encompass nutritional values, calories, direct verbal information on type and quantities, preferably specifying the relevant times of food and/or liquid intake, etc.

In several embodiments of the patient monitor system, the vital parameters, which may also be referred to herein as specific data, are or encompass cardiovascular data, heart rate (HR), heart rate variability (HRV), QT wave in the ECG, blood pulse wave, oxygen saturation, breathing rate, breathing amplitude, GSR (Galvanic Skin Response) when resistance is measured via the skin, weight, body temperature, blood pressure, or the like, and any combinations thereof.

In some embodiments of the patient monitor system, the vital parameters are or encompass the heart rate, the respiratory rate, the blood pressure and/or the body temperature. In several embodiments, the vital parameters also include oxygen saturation.

In several embodiments of the patient monitor system, the vital parameters are or encompass parameters the determination of which requires the use of one or more sensors and/or devices for data analysis or data processing.

In some embodiments of the patient monitor system, the behavior-related data is or encompasses such data the determination of which does not require the use of a sensor and/or does not require a device for data analysis or data processing.

In several embodiments of the patient monitor system, the behavior-related data is or encompasses such data that can be collected by questioning or by observation with the naked eye.

In certain embodiments of the patient monitor system, the vital parameters are not or do not encompass behavior-related data, and vice versa.

In some embodiments of the patient monitor system, the latter includes a storage device. The storage device serves to store behavior-related data and/or vital parameters of the patient collected by the collecting device and/or such data is stored on it.

Alternatively or additionally, the storage device serves to store limit values or threshold values and/or (permissible) value ranges for the behavior-related data and/or the vital parameters and/or such data is stored on it.

Alternatively or additionally, the storage device serves to store behavior-related data and/or vital parameters of the patient collected in the past. Alternatively or additionally, the memory device serves to store behavior-data and/or vital parameters obtained from patient collectives. In certain embodiments, the storage device includes such data or such data is stored in the storage device.

Data obtained from patient collectives (behavior-related data and/or vital parameters) are comparative data that were collected on a plurality of patients, preferably under the same or similar general conditions, and possibly processed statistically.

In several embodiments, the calculation unit of the patient monitor system is configured to compare the behavior-related data and/or vital parameters of the patient collected for a current period or the last observed period with the behavior-related data or vital parameters of the same patient from periods of time further in the past. Alternatively or additionally, it may be provided to bring these data into relation with each other.

The result of this comparison is evaluated using a value range and/or at least one limit value.

Where reference is made herein to a “period of time”, this may be or may encompass, for example, the period of time between two treatments, a period of 24 hours, and so on.

The value range or limit value may, in certain embodiments, be based on or defined by the data of the patient collective.

In some embodiments, the value range or limit value can be based on or defined by the data from time periods further in the past for the same patient.

Comparing and/or relating these data is also referred to herein as data analysis.

An estimation of future developments of the collected behavior-related data and/or of vital parameters of the patient and an output or display of corresponding information or a corresponding instruction may be provided. For example, static methods, trend analysis methods or predictive analytics methods can be used for this purpose.

In several embodiments, the calculation unit of the patient monitor system is configured to compare or relate the behavior-related data and/or the vital parameters of the current or most recently observed period of time with behavior-related data or vital parameters of the collective of patients. The result of this comparison may hereby be evaluated on the basis of the value range and/or on the basis of at least one limit value.

In some embodiments, the calculation unit is programmed to calculate based on the behavior-related data and/or on the vital parameters a probability of the patient suffering from diseases or secondary diseases. In addition, the calculation unit is preferably further programmed to output an instruction and/or a message or an alarm if the probability for the patient of the occurrence of a disease or secondary disease exceeds a predetermined limit value or corresponds to another predetermined criterion.

In several embodiments, the medical treatment apparatus described herein is embodied as a blood treatment apparatus, in particular as a dialysis apparatus, hemodialysis apparatus, hemofiltration apparatus, hemodiafiltration apparatus or as an apparatus for chronic renal replacement therapy, for apheresis, in particular for plasmapheresis treatment, or for whole blood adsorption treatment or as a treatment apparatus for peritoneal dialysis.

The medical treatment apparatus includes a control device or a closed-loop control device for controlling or regulating the treatment session carried out with it on the basis of changeable treatment parameters (flow rates, pressures, time periods, etc.). In some embodiments, the control device or the closed-loop control device is programmed to adapt, set for the first time, change or the like at least one of the treatment parameters on the basis of the instructions output by the calculation unit or its output device.

It is optionally provided in several embodiments that such adaptation, setting or modification, etc. of treatment parameters alternatively or additionally includes a setting or output of a range or group or list from which the attending physician may select specific treatment parameters with which the subsequent treatment or treatment session is to be carried out. Such suggested treatment parameters may be or may encompass reduced ultrafiltration rates, blood flow rates, extended duration of treatment session, etc.

The method described herein which optionally further includes transmitting the instructions to the medical treatment apparatus, takes place in some embodiments using a patient monitor system as described herein.

In several embodiments, an analysis of the patient's health status may be performed during the data analysis, in particular if the behavior-related data provide signs of deterioration in the patient's health status. If the behavior-related data yields irregularities, i.e., the patient behaves differently with respect to comparative data (his own data and/or the data of at least one patient collective), the vital parameters are specifically analyzed. The reverse procedure, in which (preferably only) the behavior-related data are collected and/or evaluated in the event of abnormalities in the vital parameters, is also encompassed by the present disclosure.

In some embodiments, based on the collected data of the patient, in particular based on data collected between two consecutive treatments or treatment sessions, in addition to adapting the treatment, recommendations for further, possibly more in-depth, diagnostic measures may be made for the patient. In several embodiments, based on the data analysis, the collected data of the patient may be visualized and compared to data of at least one patient collective and to the patient's own data history (including visually).

In some embodiments, the instruction may be a treatment adaptation that can be shown to an attending person on a display. In this regard, it may be provided that the treatment adaptation must be made by the physician/nurse. In certain embodiments, this is provided via push messages.

In several embodiments, an adaptation of the upcoming treatment or treatment session based on the collected data may already be made automatically on the machine, whereby preferably a physician/nurse must still confirm this adaptation.

In some embodiments, patients are not only patients who are already undergoing treatment, but also persons who are at increased risk of having to undergo treatment. Thus, in these embodiments, an optimal point in time at which a treatment should be carried out may be determined on the basis of the collected data of the patient.

In some embodiments, it may be provided that the behavior-related data and the vital parameters are collected automatically, in others these are entered by the patient. In several embodiments it may be provided that part of the aforementioned data is collected automatically, while another part is entered manually by the patient.

In several embodiments, the collected data (behavior-related data and/or vital parameters) are sent in real time or in a clocked manner to a calculating unit or to a database connected to the calculating unit. The data may be transmitted here as raw data or as preprocessed data. A transmission of raw data is particularly useful with a clocked connection, for example when the patient monitor system is integrated in a WLAN.

In some embodiments, the data analysis for targeted therapy may be used to avoid secondary diseases. For example, adverse physiological symptoms, such as an unstable circulation, may be understood as a secondary disease.

In several embodiments, the data analysis may be used to generate predictive models for additional disease patterns. For example, it may be provided to identify a disease pattern from the collected data (in particular vital parameters) by an “early warning score”. An “early warning score” defines certain ranges of behavior-related data and/or vital parameters or a certain course of behavior-related data and/or vital parameters or a combination thereof. Thus, a more comprehensive treatment of the patient may be carried out both in the context of the (upcoming) treatment and with regard to additional disease patterns. This may advantageously lead to a comprehensive orientation of the treatment, so that the probability of secondary diseases identified by the data analysis may be reduced.

With some embodiments according to the present disclosure, one or more of the advantages mentioned herein may be achievable, which include the following:

By using the present devices, systems, and methods, the treatment of the patient may advantageously be adapted by evaluating data that are collected between two consecutive treatments. Thus, the treatment of the patient is individually adapted, which may, for example, improve the tolerability of the treatment, which in turn contributes to increasing the well-being of the patient to be treated.

By collecting behavior-related data and/or vital parameters related to the patient's health, a holistic view of the patient may be achieved. In this way, the treatment is specifically adapted to the daily form or to health development of the patient.

All advantages achievable with the apparatuses described herein may, in certain embodiments according to the present disclosure, also be achieved undiminished with the method described herein, and vice versa.

### BRIEF DESCRIPTION OF THE DRAWINGS

In the following, the present systems, devices, and methods are described based on embodiments thereof with reference to the accompanying drawing. In the figures, the following applies:

FIG. 1 shows, schematically simplified, a fluid line diagram of a medical treatment apparatus.

FIG. 2 shows, schematically simplified, a patient monitor system in a first embodiment.

FIG. 3 shows, schematically simplified, a patient monitor system in a second embodiment.

FIG. 4 shows, schematically simplified, the course of a method in a first embodiment.

### DETAILED DESCRIPTION

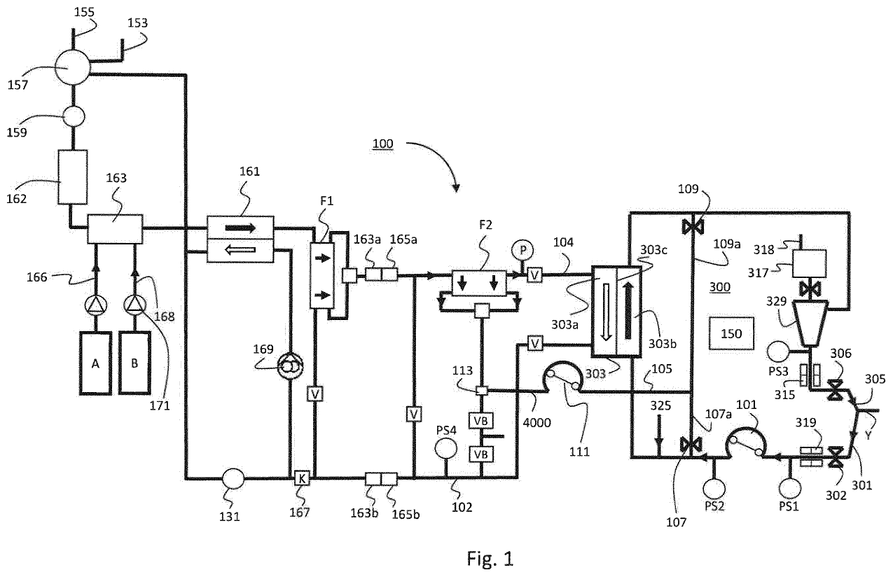

FIG. 1 shows, schematically simplified, a fluid line diagram of a medical treatment apparatus 100, here a blood treatment apparatus, connected to an extracorporeal blood circuit 300, which can be connected to the vascular system of the patient, not shown, for treatment using double-needle access, or by using, e.g., an additional Y-connector (reference numeral Y), for treatment using single-needle access, as shown in FIG. 1. The blood circuit 300 may be present, optionally in sections thereof, in or on a blood cassette.

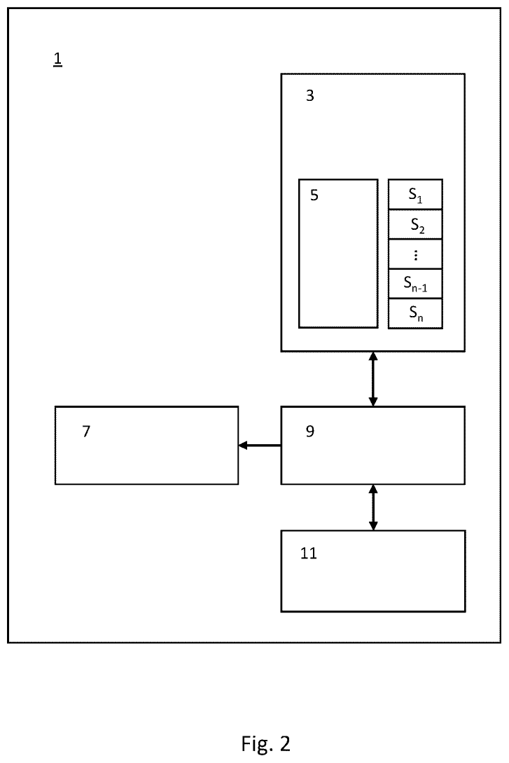

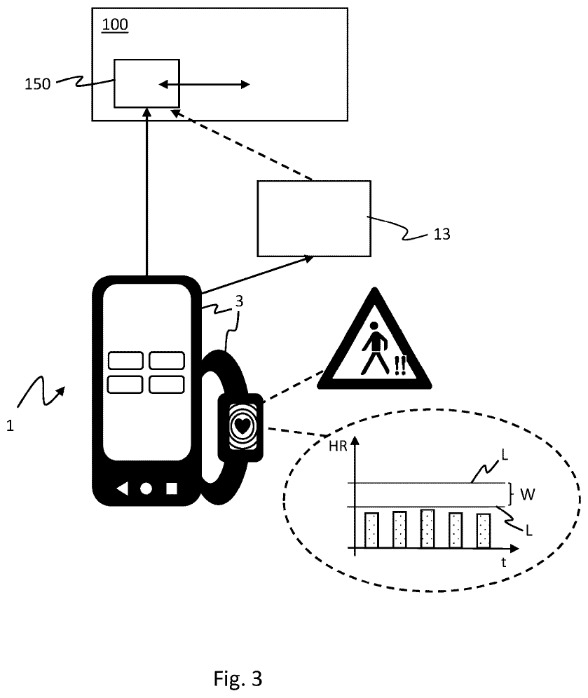

Pumps, actuators and/or valves in the area of the blood circuit 300 are connected to the treatment apparatus 100 or to a control device or closed-loop control device 150 encompassed by it. This control device or closed-loop control device 150 may be provided in order to be connected to a patient monitor system 1 (see FIGS. 2 and 3) in order to receive or call up or retrieve instructions from it. By using or based on these instructions, the treatment, in particular at least one treatment parameter, may be individually adapted to the patient to be treated.

The blood circuit 300 includes (or is connected to) an arterial patient tube clamp 302 and an arterial connection needle of an arterial section or of an arterial patient line, blood withdrawal line or first line 301. The blood circuit 300 also includes (or is connected to) a venous patient tube clamp 306 and a venous connection needle of a venous section, a venous patient line, a blood return line or a second line 305.

A blood pump 101 is provided in or at the first line 301, a substituate pump 111 is connected to a dialysis liquid inlet line 104 for conveying fresh dialysis liquid, which is filtered in a filter (F2) (substituate). A substituate line 105 may be fluidically connected to the inlet line 104. Using the substituate pump 111, substituate may be introduced by predilution, via a predilution valve 107, or by postdilution, via a postdilution valve 109, via associated lines 107a or 109a into line sections, for example into the arterial line section 301 or into the venous line section 305 (here between a blood chamber 303b of a blood filter 303 and a venous air separation chamber or venous blood chamber 329 of the blood circuit 300.

The blood filter 303 includes the blood chamber 303b connected to the arterial line section 301 and to the venous line section 305. A dialysis liquid chamber 303a of the blood filter 303 is connected to the dialysis liquid inlet line 104 leading to the dialysis liquid chamber 303a and to a dialysate outlet line 102, which guides dialysate, i.e., spent dialysis liquid, leading away from the dialysis liquid chamber 303a. Dialysis liquid chamber 303a and blood chamber 303b are separated from each other by a mostly semipermeable membrane 303c. It represents the partition between the blood side with the extracorporeal blood circuit 300 and the machine side with the dialysis liquid or dialysate circuit, which is shown in FIG. 1 to the left of the membrane 303c.

The arrangement in FIG. 1 encompasses an optional detector 315 for detecting air and/or blood. The arrangement of FIG. 1 further encompasses one or two pressure sensors PS1 (upstream of the blood pump 101) and PS2 (downstream of the blood pump 101, it measures the pressure upstream of the blood filter 303 (“pre-hemofilter”)) at the points shown in FIG. 1. Further pressure sensors may be provided, e.g., pressure sensor PS3 downstream of the venous blood chamber 329 which may serve as a bubble trap.

An optional single-needle chamber 317 is used in FIG. 1 as a buffer and/or compensating reservoir in a single-needle procedure in which the patient is connected to the extracorporeal blood circuit 300 using only one of the two blood lines 301, 305.

The arrangement of FIG. 1 also encompasses an optional detector 319 for detecting air bubbles and/or blood.

An optional addition site 325 for Heparin or for other anticoagulation medication may be provided.

On the left in FIG. 1, a mixing device 163 is shown, which provides a predetermined mixture for the respective solution from the containers A (for A-concentrate via concentrate supply 166) and B (for B-concentrate via concentrate supply 168) for use by the treatment apparatus 100. The solution contains heated water, heated, e.g., in a heat exchanger 157 (on-line, e.g., as reverse osmosis water or from bags) from the water source 155.

A pump 171, which can be referred to as concentrate pump or sodium pump, is fluidly connected to the mixing device 163 and a source of sodium, for example the container B, and/or coveys out of it.

Furthermore, FIG. 1 shows a drainage line 153 for the effluent. The optional heat exchanger 157 and a first pump or flow pump 159, which is suitable for degassing, complete the arrangement shown.

A further pressure sensor may be provided as PS4 downstream of the blood filter 303 on the water side, but preferably upstream of an optional ultrafiltration pump 131 in the dialysate outlet line 102 for measuring the filtrate pressure or membrane pressure of the blood filter 303. Additional, optional pressure measuring points P may also be provided.

Blood leaving the blood filter 303 flows through an optional venous blood chamber 329, which may include a deaeration device 318 and may be in fluid communication with the pressure sensor PS3.

The exemplary arrangement shown in FIG. 1 includes the control device or closed-loop control device 150. It may be in a wired or wireless signal connection with any of the components mentioned herein—especially or in particular with the blood pump 101—to control or regulate the treatment apparatus 100

By using the device for on-line mixing of the dialysis liquid, a variation of its sodium content, controlled by the control device or closed-loop control device 150, is possible within certain limits. For this purpose, in particular the measured values determined by the conductivity sensors 163a, 163b may be taken into account. Should an adjustment of the sodium content of the dialysis liquid (sodium concentration) or of the substituate turn out to be necessary or desired, this can be done by adjusting the conveyance rate of the sodium pump 171.

In addition, the treatment apparatus 100 includes means for conveying fresh dialysis liquid and dialysate. For this purpose, upstream of the blood filter 303, the first flow pump 159 is provided which conveys free dialysis liquid towards the blood filter 303. A first valve may be provided between the first flow pump 159 and the blood filter 303, which first valve opens or closes the inflow towards the blood filter 303 at the inlet side. A second, optional pump or flow pump 169 which conveys dialysate through the drainage line 153 is provided, e.g., downstream of the blood filter 303. A second valve may be provided between the blood filter 303 and the second flow pump 169, which second valve opens or closes the outflow at the outlet side.

Furthermore, the treatment apparatus 100 optionally includes a device 161 for balancing the flow flowing into and out of the dialyzer 303 on the machine side. The device 161 for balancing is preferably arranged in a line section between the first flow pump 159 and the second flow pump 169.

The treatment apparatus 100 further includes means, such as the ultrafiltration pump 131, for the precise removal of a volume of liquid from the balanced circuit, as predetermined by the user and/or by the control device or closed-loop control device 150.

Sensors such as the optional conductivity sensors 163a, 163b serve to determine the conductivity, which in some embodiments is temperature-compensated, as well as the fluid flow upstream and downstream of the dialyzer 303.

Temperature sensors 165a, 165b may be provided as one or a plurality thereof. Temperature values supplied by them may be used to determine a temperature-compensated conductivity.

A leakage sensor 167 is optionally provided.

Further flow pumps in addition or alternatively to, e.g., the one with the reference numeral 169 may also be provided.

A number of optional valves are each denoted with V in FIG. 1; by-pass valves with VB.

The control device or closed-loop control device 150 may determine for example the electrolyte balance and/or liquid balance based on the measured values from the aforementioned optional sensors.

Filters F1 and F2 can be connected in series.

Even when using non-pure water, the filter F1 exemplarily serves herein to generate sufficiently pure dialysis liquid by the mixing device 163, which then flows through the blood filter 303, e.g., using the countercurrent principle.

The filter F2 exemplarily serves herein to generate sterile or sufficiently filtered substituate from the sufficiently pure dialysis liquid leaving the first filter F1, by filtering, e.g., pyrogenic substances. This substituate may then be safely added to the extracorporeally flowing blood of the patient and thus ultimately to the patient's body.

The treatment apparatus 100 is optionally shown in FIG. 1 as a device for hemo(dia)filtration. However, hemodialysis apparatuses, a blood treatment apparatus for peritoneal dialysis or other medical treatment apparatuses are also covered by the present disclosure, although not specifically represented in a figure. The treatment apparatus may thus be located at the patient's home or in a dialysis center or hospital.

A port 113 inside the treatment apparatus 100 may be provided, for example to enable rinsing or priming the extracorporeal blood circuit 300.

The present disclosure is not limited to the embodiment described above; this only serves for illustration.

The arrows shown in FIG. 1 generally indicate the flow direction in FIG. 1.

FIG. 2 shows, schematically simplified, a patient monitor system 1 in a first embodiment. With this, patient-related data (behavior-related data and/or vital parameters) may be collected, preferably between two treatments or treatment sessions.

The patient monitor system 1 includes a collecting device 3, which in turn includes a number of sensors S₁ to Sₙ and/or an optional input device 5.

The herein exemplary n sensors may in particular be provided to automatically, for example continuously or at certain time intervals, or initiated by the calculation unit 9, which is also encompassed by the patient monitor system 1, to take measurements, in particular of vital parameters (e.g., cardiovascular data, HR, HRV, QT wave, temperature, etc.) and/or collect behavior-related data of the patient (e.g., regarding exercise, sleep, etc.).

The input device 5 may in particular be provided in order to collect data entered manually by the patient, for example because they cannot be collected automatically with the sensors. Manually entered data may include some vital parameters (e.g. weight, etc.) as well as behavior-related data (e.g., liquid and/or food intake, medication intake, etc.). The input device 5 may optionally be provided in a common device with the collecting device 3, for example as push buttons or touchscreen of a wearable device, for example a wristwatch. The input device 5 may, however, also be implemented or realized via a further device. For example, the input device 5 may be provided on or in a smartphone, tablet or keyboard of the home computer of the patient or of the attending person. A wireless connection may thus take place between the collecting device 3 and the device on which the input device 5 is optionally implemented. This may be done via a home network, a radio link or a Bluetooth connection. The data collected by the collecting device 3 may thus be changed or supplemented via the input device 5.

By the calculation unit 9 or by another device, the above-mentioned data may be stored in or on an optionally available storage device 11, or the storage may be initiated by it. The calculation unit 9 may also be part of the collecting device 3, or it may be designed decentral to it. Thus, the calculation unit 9 may be arranged on a further mobile terminal, a server, which is arranged in a monitoring station for the attending persons, or designed in a decentralized manner. The calculation unit 9 may also be constructed in a cloud-based manner.

The storage device 11 is or includes a, in particular non-volatile, storage medium, in particular in the form of a machine-readable carrier, in particular in the form of a flash-based memory chip, a memory card, EEPROM, FRAM (Ferroelectric RAM) or SSD (Solid-State-Drive), or a combination thereof, in particular with electronically readable control signals. Likewise, the storage device 11 may also be located in the collecting device 3, a further mobile terminal of the patient or of the attending person, a decentralized server or designed as a cloud-based memory.

Limit or threshold values and/or (permissible) value ranges for the behavior-related data and/or vital parameters may be stored in the storage device 11.

Corresponding data of the patient (behavior-related data and/or vital parameters) already collected in the past by the collecting device 3 may also be stored in the storage device 11. Alternatively or in addition, behavior-related data and/or vital parameters obtained from at least one patient collective may be stored in the storage device 11.

In several embodiments, the calculation unit 9 is further configured to generate instructions by comparing the current data (behavior-related data and/or vital parameters) of the patient detected by the collecting device 3 with comparison values (behavior-related data and/or vital parameters) stored in the storage device 11. The instructions, for example, be used by a control device or closed-loop control device 150 of a medical treatment apparatus 100 (see FIG. 3) to individually adjust treatment parameters for the upcoming treatment of the patient. The comparison values may be, or may encompass, limit or threshold values, (permissible) value ranges, data of the patient determined in the past and/or data of the patient collective.

The instructions may for this purpose be output by the output device 7, for example to the control device or closed-loop control device 150 of the medical treatment apparatus 100.

In several embodiments, the instructions are then or only then generated and/or output by the output device 7 if at least the behavior-related data of the patient, or a part of these data, on the one hand, and the vital parameters of the patient, or a part of these vital parameters, on the other hand, do not lie in a value range (W) predetermined for them and/or lie beyond a limit value (L) predetermined for them (see FIG. 3).

FIG. 3 shows, schematically simplified, a patient monitor system 1 in a second embodiment. In this embodiment, the collecting device 3 is present on two separate devices, here, for example, a smartphone and a wristband connected and/or in signal communication thereto.

The sensors S₁, . . . , Sₙ (see FIG. 2) can be distributed on the wristband and the smartphone, and input by the patient can also be possible via the wristband display or via the smartphone display. It is also encompassed that this should be possible only on one of said devices.

Corresponding communication devices, suitably programmed and/or configured for this purpose, are also encompassed.

In the example of FIG. 3, the behavior-related data between two blood treatments, in particular dialysis treatments, show that the patient moves very little, represented by the triangular warning sign with a pedestrian. This is followed by a targeted analysis of his heart rate (see diagram heart rate [HR] over time [t]). If, as in the example shown, this also shows a very low value, in particular a value which is below the lower limit value L or outside the permissible value range W, then, in the present case, a possible circulatory instability may be concluded. Before the next treatment, this course of events, for example in comparison to the average values of previous treatments can be shown to the attending physician. Subsequently, an instruction generated by the calculation unit 9 may be sent to the control device or closed-loop control device 150 of the treatment apparatus 100 or to the attending person (e.g., that there is an indication for further examinations or the like). For this specific example, an attenuated UF profile could be suggested or the re-determination of the dry weight with a suitable diagnostic device (e.g., BCM (short for Body Composition Monitor)) could be recommended. Preferably, changes in the treatment parameters are displayed to the attending person for confirmation. The instruction may be displayed directly on the treatment apparatus itself, for example via its display, or on a remote terminal. For example, an overview of upcoming treatments and a recommendation for treatment adjustment may be presented to the attending person.

In another example, if the behavior-related data, which could herein also be referred to as non-specific data, show that the patient sleeps comparatively little and possibly restlessly between two treatment sessions, and if the specific data show that the blood pressure is higher than the patient's comparison values, the physician is shown the treatment proposal as instructions to increase the amount of water to be removed during treatment sessions in the course of the following dialysis treatment.

If, in yet another example, the behavior-related data show that the patient's sleep and/or movement behavior on the day after dialysis treatment differs significantly from that on the day before treatment, it can be concluded that the patient is excessively stressed by the treatment parameters selected last. Before the next treatment, this course in comparison to average values is for example displayed or shown to the physician. Depending on specific treatment parameters, the physician will be given a recommendation to readjust the medication or to check and adjust the composition of the dialysis liquid.

If, on the other hand, the behavior-related data show in another example that the patient changes their behavior (amount of movement, proportion of time spent in motion, standing, sitting) towards calmer behavior and if the specific data such as temperature and heart rate show an increase, this is communicated and visualized to the physician before treatment. The change must deviate from the characteristic daily course by a certain amount. This may provide the doctor with information about an emerging infection process in the patient and a recommendation for further measures (depending on the severity, such as blood tests) is given.

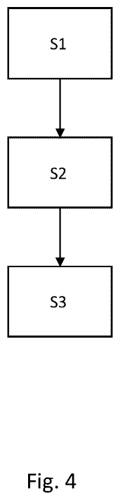

FIG. 4 shows schematically simplified a first embodiment of the course of a method for the preparation of a treatment or treatment session of a patient. The treatment or treatment session is performed by using a medical treatment apparatus. The preparation of a treatment or a treatment session takes place at a time during which the patient to be treated is not yet connected to the treatment apparatus 100, in particular not yet connected to its extracorporeal blood circuit 300.

In the example of FIG. 4, step S1 represents a detection of behavior-related data, e.g., data on sleep behavior, movement behavior (e.g. using a pedometer), regular medication, etc. In this, it may be provided to detect these data via sensors automatically and/or initiated by the calculation unit 9 or to have them entered by the patient or the attending person using the input device 5.

In the example of FIG. 4, step S2 represents a detection of vital parameters, e.g., cardiovascular data (e.g., HR, HRV, QT wave, or similar), weight, temperature, etc. In this, it may likewise be provided to detect these data via sensors automatically and/or initiated by the calculation unit 9 or to have them entered by the patient or the attending person using the input device 5.

In both step S1 and step S2 it is provided to initiate or perform the detection at at least one point in time when the patient is not being treated by the medical treatment apparatus (100) in a treatment session, i.e., for example between two successive treatments or treatment sessions by using the medical treatment apparatus (100).

Step S3 represents the outputting of instructions if both the behavior-related data of the patient on the one hand and the vital parameters of the patient on the other hand do not lie within a value range (W) predetermined for them and/or lie beyond a limit value (L) predetermined for them.

In certain embodiments, it may be provided that the outputting of instructions is or encompasses transmitting these instructions to the treatment apparatus 100, in particular to the control device or closed-loop control device thereof. Alternatively or in addition, it may be provided to output the instructions on a display device 13 in order to request confirmation from the attending person. The display device 13 may be or include a display device, e.g., a display of the patient monitor system 1 or a display device, e.g., a screen or display of the treatment apparatus 100 (see FIG. 3).

### LIST OF REFERENCE NUMERALS

1 patient monitor or monitoring system
3 collecting device
5 input device
7 output device
9 calculation unit
11 storage device
13 display device
100 medical treatment apparatus
101 blood pump
102 dialysis outlet line
104 dialysis liquid inlet line
105 substituate line
107 predilution valve
107a line corresponding or assigned to the predilution valve
109 postdilution valve
109a line corresponding or assigned to the postdilution valve
111 substitute pump
113 port
131 ultrafiltration pump
150 control device or closed-loop control device
153 drainage line or discharge line
155 water source
157 heat exchanger
159 first flow pump
161 device for balancing
163 mixing device
163a conductivity sensor
163b conductivity sensor
165a temperature sensor
165b temperature sensor
166 concentrate supply
167 leakage sensor
168 concentrate supply
169 second flow pump
171 natrium pump
300 extracorporeal blood circuit
301 first line (arterial line section)
302 (first) tube clamp,
arterial patient tube clamp
303 blood filter or dialyzer
303a dialysis liquid chamber
303b blood chamber
303c semipermeable membrane
305 second line (venous line section)
306 (second) tube clamp,
venous patient tube clamp
315 detector
317 single-needle chamber
318 deaeration device
319 detector
325 addition site for Heparin
329 venous blood chamber (optional)
A container
B container
F1 filter
F2 filter
L limit value; limit
W value range
P pressure measuring points
PS1 arterial pressure sensor (optional)
PS2 arterial pressure sensor (optional)
PS3 pressure sensor (optional)
PS4 pressure sensor for measuring the filtrate pressure (optional)
S₁, . . . , Sₙ sensors
V valve(s)
VB bypass valves
Y Y connector

1-15. (canceled)

16. A patient monitor system, comprising:
at least one collecting device configured and/or programmed to collect:
behavior-related data of a patient; and/or
vital parameters of the patient;
an output device; and
a calculation unit, programmed to:
receive the behavior-related data and/or the vital parameters collected by the at least one collecting device;
generate instructions in order to control or regulate a medical treatment apparatus; and
output the instructions using the output device,
wherein the calculation unit is further programmed to generate the instructions and/or to have the instructions output by the output device when the behavior-related data of the patient, or part of the behavior-related data, do not lie in a predetermined behavior-related data value range and/or lie beyond a predetermined behavior-related data limit value, and/or the vital parameters of the patient, or part of the vital parameters do not lie in a predetermined vital parameter value range and/or lie beyond a predetermined vital parameter limit value.

17. The patient monitor system according to claim 16, wherein the calculation unit is programmed to initiate or trigger the collecting device in order to collect the behavior-related data and/or vital parameters using the collecting device at a point in time at which the patient is not being treated by the medical treatment apparatus in a treatment session, or between two successive treatment sessions by the medical treatment apparatus.

18. The patient monitor system according to claim 16, wherein the calculation unit is programmed to detect or evaluate the vital parameters, or a part thereof, only in the event that the calculation unit has recognized that the detected behavior-related data, or a part thereof, do not lie in the predetermined behavior-related data value range and/or lie beyond the predetermined behavior-related data limit value, and/or wherein the calculation unit is programmed to collect or evaluate the behavior-related data, or a part thereof only in the event that the calculation unit has recognized that the collected vital parameters, or a part thereof, do not lie in the predetermined vital parameter value range and/or lie beyond the predetermined vital parameter limit value.

19. The patient monitor system according to claim 16, wherein the collecting device is designed as a portable device.

20. The patient monitor system according to claim 19, wherein the collecting device is designed as a wristband, wristwatch, mobile phone, wearable, accessory, as a garment, or part thereof or combinations thereof.

21. The patient monitor system according to claim 16, wherein
the behavior-related data are or encompass sleep data, movement data, activity data, a parameter of a medication intake, and/or data on food and/or fluid intake; and wherein
the vital parameters are or encompass cardiovascular data, heart rate, heart rate variability, QT wave, blood pulse wave, oxygen saturation, respiratory rate, respiratory amplitude, galvanic skin response, weight, body temperature and/or blood pressure.

22. The patient monitor system according to claim 21, wherein the activity data comprises a number of steps taken per day.

23. The patient monitor system according to claim 21, wherein the parameter of the medication intake comprises a regularity of the medication intake.

24. The patient monitor system according to claim 16, further comprising a storage device for storing: the behavior-related data and/or vital parameters of the patient collected by the collecting device, behavior-related data and/or vital parameters of the patient collected in the past, behavior-related data and/or vital parameters obtained from patient collectives, limit values, and value ranges.

25. The patient monitor system according to claim 16, wherein the calculation unit is configured to compare the behavior-related data and/or the vital parameters of a current or most recently viewed period with the behavior-related data and/or vital parameters of past time periods of the patient, and wherein the result of the comparison is evaluated based on the behavior-related data value range, the behavior-related data limit value, the vital parameter value range, and/or the vital parameter limit value.

26. The patient monitor system according to claim 16, wherein the calculation unit is configured to compare the behavior-related data and/or the vital parameters of a current or most recently viewed period with the behavior-related data or the vital parameters of a patient collective, and wherein the result of the comparison is evaluated based on the behavior-related data value range, the behavior-related data limit value, the vital parameter value range, and/or the vital parameter limit value

27. The patient monitor system according to claim 16, wherein the calculation unit is programmed to calculate a patient's probability of an occurrence of diseases or secondary diseases based on the behavior-related data and/or the vital parameters, and is further programmed in order to output an instruction if the patient's probability of the occurrence of diseases or secondary diseases exceeds a predetermined disease limit value.

28. A display device for displaying at least one instruction for controlling or regulating a medical treatment apparatus, wherein the display device is part of a patient monitor system according to claim 16 and/or is in signal communication with the output device of a patient monitor system according to claim 16.

29. A medical treatment apparatus with the display device according to claim 28 and/or in signal communication with the display device according to claim 28.

30. A medical treatment apparatus having a patient monitor system according to claim 16 and/or in signal communication with the patient monitor system according to claim 16.

31. The medical treatment apparatus according to claim 29, embodied as a blood treatment apparatus.

32. The medical treatment apparatus according to claim 30, embodied as a dialysis apparatus, hemodialysis apparatus, hemofiltration apparatus, hemodiafiltration apparatus or as an apparatus for chronic renal replacement therapy, for apheresis, or for whole blood adsorption treatment or as a treatment apparatus for peritoneal dialysis.

33. The medical treatment apparatus according to claim 31, embodied for plasmapheresis treatment.

34. The medical treatment apparatus according to claim 29, comprising a control device or closed-loop control device which is programmed to adjust or set at least one treatment parameter based on the instructions outputted by the calculation unit.

35. A method for preparing a treatment or treatment session of a patient to be performed by a medical treatment apparatus, the method comprising:
detecting behavior-related data at least one point in time at which the patient is not being treated by the medical treatment apparatus in a treatment session, or between two successive treatments or treatment sessions by the medical treatment apparatus.
collecting vital parameters at at least one point in time at which the patient is not being treated by the medical treatment apparatus in the treatment session, or between two successive treatments or treatment sessions by the medical treatment apparatus.
outputting instructions if or when the behavior-related data of the patient do not lie in a predetermined behavior-related data value range and/or lie beyond a predetermined behavior-related data limit value, and/or the vital parameters of the patient do not lie in a predetermined vital parameter value range and/or lie beyond a vital parameter limit value.

36. The method according to claim 34, wherein the method is carried out using a patient monitor system comprising:
at least one collecting device configured and/or programmed to collect:
the behavior-related data of the patient; and/or
the vital parameters of the patient;
an output device; and
a calculation unit, programmed to:
receive the behavioral data and/or the vital parameters collected by the at least one collecting device;
generate the instructions in order to control or regulate the medical treatment apparatus; and
output the instructions using the output device;
wherein the calculation unit is further programmed to generate the instructions and/or to have the instructions output by the output device when the behavior-related data of the patient, or part of the behavior-related data, do not lie in the predetermined behavior-related data value range and/or lie beyond the predetermined behavior-related data limit value, and/or the vital parameters of the patient, or part of the vital parameters do not lie in the predetermined vital parameter value range and/or lie beyond the predetermined vital parameter limit value.
